(19) United States
(12) Patent Application Publication
(10) Pub. No.: US 2026/0114739 A1
(43) Pub. Date: Apr. 30, 2026

(54) WRISTBAND DEVICE AND SYSTEM WITH GESTURE RECOGNITION FUNCTION AND CONTINUOUS BLOOD PRESSURE MONITORING FUNCTION

(71) Applicant: SOUTHEAST UNIVERSITY, Wuxi (CN)

(72) Inventors: Zhen ZHU, Wuxi (CN); Shuangye XU, Wuxi (CN); Xiaoyu HUANG, Wuxi (CN); Juncheng FAN, Wuxi (CN)

(73) Assignee: SOUTHEAST UNIVERSITY, Wuxi (CN)

(21) Appl. No.: 19/160,062

(22) PCT Filed: Feb. 6, 2024
(86) PCT No.: PCT/CN2024/076234

§ 371 Date: Aug. 27, 2025

(30) Foreign Application Priority Data
Jan. 15, 2024 (CN) 202410054119.0

(51) Int. Cl. A61B 5/021; A61B 5/00; A61B 5/026; A61B 5/0536

(52) U.S. Cl. CPC A61B 5/02108; A61B 5/02141; A61B 5/026; A61B 5/0536; A61B 5/681; A61B 5/7225; A61B 5/7228; A61B 5/7264; A61B 5/742; A61B 2560/0214; A61B 2560/0223; A61B 2560/0468; A61B 2562/046; A61B 2562/227

(57) ABSTRACT

A wristband device and system with a gesture recognition function and a continuous blood pressure monitoring function are provided. The wristband device acquires wrist impedance distribution data and pulse impedance waveform data of the radial artery in two functional modes by means of a multiplexed electrode array in a wearable wristband and converts bioimpedance data into human gesture and blood pressure information by means of an algorithm deployed in the system to realize monitoring of human physiological conditions. The device and system can extract weak bioimpedance signals by means of the low-cost and simple electrode array and improve the monotiling sensitivity of wrist impedance signals by optimizing the electrode configuration; and two functions are integrated in the same system by means of the multiplexed electrode array, such that the sensor size, complexity and integration difficulty are reduced.

### CROSS REFERENCE TO THE RELATED APPLICATIONS

This application is the national phase entry of International Application No. PCT/CN2024/076234, filed on Feb. 6, 2024, which is based upon and claims priority to Chinese Patent Application No. 202410054119.0, filed on Jan. 15, 2024, the entire contents of which are incorporated herein by reference.

### TECHNICAL FIELD

The invention relates to bioimpedance measurement technology and gesture recognition and continuous pulse and blood pressure measurement technology for monitoring of human physical conditions, particularly discloses an electrical impedance-based wearable wristband device and system with a gesture recognition function and a continuous blood pressure monitoring function, and belongs to the technical field of measurement and testing.

### BACKGROUND

In recent years, the user-friendly design of electronic equipment is receiving more and more attention, and wearable electronic equipment such as smart wristbands and AR/VR glasses become extremely popular. Users can carry around the wearable electronic equipment and use them for human-machine interaction, daily health monitoring, and other purposes.

The optimization of the ease of use and conform of the electronic equipment in use is highly valued, and gesture recognition is an important approach for realizing human-machine interaction. Traditional gesture recognition techniques often rely on computer vision, flexible angular displacement sensors and myographic sensors. The gesture recognition technique based on computer vision is limited by the light environment and the calculation capacity and thus cannot satisfy the wearable requirement. The flexible angular displacement sensors are often integrated in gloves to be used by users, but the gloves wrapping around the hands of users hinder natural motions of the hands. Electrodes of the myographic sensors require a large contact area with skin, making it difficult to integrate the myographic sensors in small wearable equipment. In view of this, the requirement for a gesture recognition technique that has a low requirement for the calculation capacity and is friendly to users and beneficial to integration is raised, and the gesture recognition technique based on electrical impedance exactly satisfies such a requirement.

One important function of portable wearable equipment is daily health monitoring, and the blood pressure is an important indicator for health monitoring. Traditionally, inflatable cuff sphygmomanometers are used for monitoring the blood pressure, but such sphygmomanometers have a large size and are not portable, it takes a long time to pressurize and depressurize the arm every time the blood pressure is measured, and continuous blood pressure monitoring cannot be realized. Smart wristbands on the present market typically adopt a blood pressure monitoring scheme based on pulse waves, and some wristbands extract pulses by means of photoplethysmography (PPG) sensors integrated in the wristbands to calculate the blood pressure. However, such a method has a limited accuracy due to the influence of ambident light and skin color, and compared with the impedance-based method, has a higher cost. Therefore, the continuous pulse and blood pressure monitoring scheme based on electrical impedance is an alternative scheme that has a lower cost and is easy to popularize.

Wearable equipment is becoming increasingly important in our daily life, so it is of particular importance to make a breakthrough in improvement of the sensing method so as to improve the monitoring accuracy, expand the monitoring range and reduce the cost and size of wearable equipment. The electrical impedance sensing technique uses electrodes as sensors, implements sensing by applying excitation currents and acquiring response voltages, and has the features of low detection cost and quick, accurate and easy detection. The ground-breaking application combining the bioelectrical impedance sensing technique and wearable equipment is expected to reduce the cost and size of the wearable equipment, improve the monitoring accuracy of the wearable equipment or expand the monitoring range of the wearable equipment.

Electrodes are an important factor that affects the accuracy of bioelectrical impedance measurement data. The electrodes of existing bioelectrical impedance measurement devices are distributed according to a fixed area to be detected, and the number of sensing electrodes is small. Some devices, the electrodes of which are distributed around the limbs, have a good monitoring effect only on a sectional area defined by the electrodes and cannot monitor the change of impedance beyond the electrode area, thus being not suitable for monitoring complex human physical conditions. Therefore, existing electrode design cannot accurately measure bioelectrical impedance for gesture recognition, making it impossible to integrate the gesture recognition technique with wearable equipment.

When a weak impedance change signal of the radial artery is monitored, the position of the radial artery cannot be quickly determined due to the difference in the shape of different human wrists, and a deviation of the position of the radial artery will severely affect an actual measurement result. For a sensor array formed by a small number of electrodes, the position of the sensor array often needs to be manually adjusted to be aligned with the radial artery to fulfill an optimal monitoring effect, and if the position of the sensor array deviates from the radial artery, an actual measurement result will be severely affected, which is not beneficial for the use of users, but also reduces the continuous pulse and blood pressure monitoring accuracy. Hence, the electrode design of existing bioelectrical impedance measurement techniques cannot satisfy the requirement for continuous pulses and blood pressure measurement of wearable equipment.

Considering that existing gesture recognition techniques based on electrical impedance and existing pulse and blood pressure monitoring techniques based on electrical impedance cannot yet be well integrated with wearable equipment, the invention aims to provide a wearable wristband device and system with a gesture recognition function and a continuous blood pressure monitoring function to overcome the defects mentioned above.

### SUMMARY

The objective of the invention is to overcome the abovementioned defects by providing a wearable wristband device and system with a gesture recognition function and a continuous blood pressure monitoring function, which combine the electrical impedance sensing technique and wearable equipment to realize human-machine interaction and health monitoring, thus providing a scheme for user friendliness, sensing front-end simplification and miniaturized integration of wearable daily health monitoring equipment, thus expanding the application of the electrical impedance sensing technique in the health monitoring field and solving the technical problem that the existing gesture recognition technique based on electrical impedance and the existing pulse and blood pressure monitoring technique based on electrical impedance cannot be well integrated with wearable equipment.

To fulfill the above objective, the invention adopts the following technical solutions:

A wristband device with a gesture recognition function and a continuous blood pressure monitoring function includes a wearable wristband, a PCB and a display screen, wherein the wearable wristband includes at least two wristband assemblies, each wristband assembly includes an assembly body and at least two contact sensing electrodes inlaid in an inner wall of the assembly body, the at least two contact sensing electrodes inlaid in a same assembly body are arranged vertically, and each contact sensing electrode is configured to be in an excitation mode or an acquisition mode; the PCB is used for realizing switching between a gesture recognition functional mode and a continuous blood pressure monitoring functional mode, applying an excitation current to an excitation current transmitter formed by two electrodes respectively selected from two adjacent wristband assemblies and acquiring response voltage signals fed back by response voltage receivers formed by electrodes respectively selected from other adjacent wristband assemblies in the gesture recognition functional mode, modulating the response voltage signals acquired in the gesture recognition functional mode into wrist impedance distribution data, applying an excitation current to an electrode pair on one wristband assembly in the vicinity of a radial artery and acquiring response voltage signals fed back by electrode pairs on other wristband assemblies in the continuous blood pressure monitoring functional mode, modulating an optimal response voltage acquired in the continuous blood pressure monitoring functional mode into pulse impedance waveform data of the radial artery in each cardiac cycle, transmitting the wrist impedance distribution data or the pulse impedance waveform data of the radial artery in each cardiac cycle to an upper computer, and receiving a gesture recognition and classification result or a predicted blood pressure fed back by the upper computer, and the display screen is used for visualizing a heat rate of a user, the pulse impedance waveform data of the radial artery in each cardiac cycle and the gesture recognition and classification result or the predicted blood pressure received by the PCB.

As a further optimized scheme of the wristband device with a gesture recognition function and a continuous blood pressure monitoring function, the optimal response voltage signal acquired in the continuous blood pressure monitoring functional mode is obtained as follows: complex amplitudes of the response voltage signals fed back by the electrode pairs on other wristband assemblies are extracted to be used as bioimpedance representations of the radial artery and tissue around, the complex amplitudes of the response voltage signals fed back by the electrode pairs on other wristband assemblies are acquired continuously, the complex amplitudes of the response voltage signals that change continuously with time are taken as pulse and blood flow representation waveforms, amplitudes of the pulse and blood flow representation waveforms obtained according to the response voltage signals acquired by the electrode pairs on other wristband assemblies are compared, and the response voltage signal corresponding to the pulse and blood flow representation waveform with a maximum amplitude and a maximum pulse wave signal peak-to-peak value is selected as the optimal response voltage signal.

As a further optimized scheme of the wristband device with a gesture recognition function and a continuous blood pressure monitoring function, the at least two wristband assemblies are connected by perforation or snap fit to form the wearable wristband capable of encircling a wrist, and the assembly body is made from, but not limited to, nylon or silicone; and the contact sensing electrodes are hemispherical electrodes, square electrodes, SMT electrodes or button electrodes.

As a further optimized scheme of the wristband device with a gesture recognition function and a continuous blood pressure monitoring function, the PCB includes an excitation source module, a signal demodulation module, a multiplexing module, a control module, a communication module and a power supply module, wherein the excitation source module is used for applying an excitation current to the contact sensing electrodes in the excitation mode, the signal demodulation module is used for receiving response voltages fed back by the contact sensing electrodes in the acquisition mode, demodulating the response voltage signals acquired in the gesture recognition functional mode into the wrist impedance distribution data, and demodulating the optimal response voltage signal acquired in the continuous blood pressure monitoring functional mode into the pulse impedance waveform data of the radial artery in each cardiac cycle; the multiplexing module is used for connecting any one excitation current transmitter to the excitation source module and connecting the response voltage receivers to the signal demodulation module in the gesture recognition functional mode until the excitation current is applied to all the excitation current transmitters, and connecting the electrode pair of the wristband assembly in the vicinity of the radial artery to the excitation source module and connecting the electrode pairs of other wristband assemblies to the signal demodulation module in the continuous blood pressure monitoring functional mode; the control module is used for controlling gating of channels in the multiplexing module, communication between a communication module and the display screen, and start and stop of the excitation source module and the signal demodulation module; the communication module is used for transmitting the wrist impedance distribution data or the pulse impedance waveform data of the radial artery in each cardiac cycle to an upper computer, receiving the gesture recognition and classification result or the predicted blood pressure fed back by the upper computer, and transmitting the gesture recognition and classification result or the predicted blood pressure to the display screen; and the power supply module is used for providing an operating voltage and power for full-load operation of the PCB.

As a further optimized scheme of the wristband device with a gesture recognition function and a continuous blood pressure monitoring function, the excitation source module includes a waveform lookup table, a digital-to-analog converter and a voltage-controlled current source, wherein the waveform lookup table is used for generating a unipolar sinusoidal voltage signal; the digital-to-analog converter is used for converting the unipolar sinusoidal voltage signal into an analog signal and outputting the analog signal; and the voltage-controlled current source is used for converting the analog signal output by the digital-to-analog converter into a differential current signal and outputting the differential current signal.

As a further optimized scheme of the wristband device with a gesture recognition function and a continuous blood pressure monitoring function, the signal demodulation module includes a differential amplifier, an analog-to-digital converter and a data demodulator, wherein the differential amplifier is used for performing differential amplification on the received response voltage signal and outputting a differential signal; the analog-to-digital converter is used for converting the differential signal output by the differential amplifier into a single-ended signal, converting the single-ended signal into a digital signal and outputting the digital signal; and the data demodulator is used for extracting the wrist impedance distribution data or the pulse impedance waveform data of the radial artery in each cardiac cycle from the digital signal output by the analog-to-digital converter.

As a further optimized scheme of the wristband device with a gesture recognition function and a continuous blood pressure monitoring function, the multiplexing module is formed by four multiplexer chips, common terminals of the four multiplexer chips are respectively connected to two output terminals of the voltage-controlled current source and two input terminals of the differential amplifier, each contact sensing electrode is electrically connected to a gateable channel of one multiplexer chip, and an address line for controlling gating of the channels of the four multiplexer chips is connected to the control module.

As a further optimized scheme of the wristband device with a gesture recognition function and a continuous blood pressure monitoring function, the display screen is fixed in a mechanical groove, the mechanical groove is seamlessly adhered to a top of the PCB with a laminating adhesive to realize overlapped assembly, and the wearable wristband is electrically connected to the PCB.

A system with a gesture recognition function and a continuous blood pressure monitoring function includes the wristband device and a PC terminal in wireless communication with the wristband device, wherein the PC terminal includes a communication control module, a pulse feature extraction module, a blood pressure prediction module, a wrist cross-section image operation module, a gesture classification module and a result display module, wherein the communication control module is used for controlling the communication between the PC terminal and the wristband device to be enabled or disenabled, receiving wrist impedance distribution data or pulse impedance waveform data of a radial artery in each cardiac cycle transmitted from the wristband device, and transmitting a gesture recognition and classification result or a predicted blood pressure calculated by the PC terminal back to the wristband device; the pulse feature extraction module is used for converting the pulse impedance waveform data of the radial artery in each cardiac cycle into pulse feature data to construct a pulse feature set; the blood pressure prediction module is used for training a pre-deployed neural network regressor according to the pulse feature set, wherein the neural network regressor, after being trained, calculates the predicted blood pressure according to the pulse impedance waveform data of the radial artery in each cardiac cycle transmitted from the wristband device; the wrist cross-section image operation module is used for converting the wrist impedance distribution data transmitted from the wristband device into a wrist cross-section impedance change distribution image; the gesture classification module is used for training a pre-deployed neural network classifier according to the wrist cross-section impedance change distribution image, wherein the neural network classifier, after being trained, predicts the gesture recognition and classification result according to the real-time wrist impedance distribution data transmitted from the wristband device; and the result display module is used for visualizing the wrist impedance distribution data or the pulse impedance waveform data of the radial artery transmitted from the wristband device, and visualizing the gesture classification result predicted by the gesture classification module or the predicted blood pressure calculated by the blood pressure prediction module.

As a further scheme of the system with a gesture recognition function and a continuous blood pressure monitoring function, the pulse feature set constructed by the pulse feature extraction module includes pulse feature data and a user blood pressure calibrated at a same time when the pulse impedance waveform data of the radial artery acquired, and the pulse feature data include, but not limited to, maximum slopes, impedance amplitudes and time intervals of a waveform segment from a starting point to a dominant wave peak of a pulse and blood flow representation waveform, a waveform segment from the dominant wave peak to a dicrotic wave trough of the pulse and blood flow representation waveform, a waveform segment from the dicrotic wave trough to an end point of the pulse and blood flow representation waveform, and an arca defined by an impedance amplitude axis and a time axis.

Compared with the prior art, the technical solutions of the invention have the following advantages and beneficial effects:
(1) According to the wristband device provided by the invention, electrodes are arranged in multiple rows and then inlaid in assemblies of the wearable wristband, such that the distribution density of the electrodes on the human body surface is increased, the contact area between the electrodes and a to-be-measured human part is enlarged, and an effective sensing area is expanded from a single cross-section to an area between two cross-sections to adapt to monitoring of complex human conditions; by detecting the electrical impedance of the wrist in real time, gesture recognition and pulse and blood pressure monitoring can be realized, these two functions can be realized by one device by means of ingenious multiplexing of the electrodes, and the sensor size, complexity and integration difficulty are reduced.
(2) By means of the design of the electrode array in the invention, the position of the wristband does not need to be adjusted anymore when the wristband is used for monitoring the pulse and blood pressure, the electrodes distributed in the vicinity the radial artery at a high density can completely cover the area in the vicinity of the radial artery, a plurality of electrode pairs formed in the area in the vicinity of the radial artery can be used for sensing, the electrode pair with the maximum measurement sensitivity can be determined by comparing peaks of pulse wave representation waveforms of multiple electrode pairs to fulfill an optimal sensing effect, and only two electrode pairs with the best sensing effect are selected, such that the using process is greatly simplified, the measurement accuracy is guaranteed, the position of the wristband does not need to be adjusted, and user experience is improved; in addition, compared gloves integrated with electrodes, a watch or wristband integrated with electrodes is more practical, comfortable, and particularly suitable for long-term wearing and daily use.
(3) In the invention, the design of arc protrusions on the electrodes ensure that the electrodes can be in tight contact with human skin when the wristband is tightened, thus improving the measurement stability and accuracy, and also improving the wearing experience; compared with traditional planar electrodes, the electrodes adopting such a design are more effective and have better performance particularly when used for measuring weak impedance change signals used for pulse or blood pressure measurement; weak bioimpedance signals can be extracted by means of a low-cost and simple electrode array, and the monitoring sensitivity of wrist impedance signals is improved by optimizing the electrode configuration.
(4) Different from traditional disposable electrocardioelectrodes, the electrodes in the electrode array in the invention can be used repeatedly and can be integrated in fabric to be worn, such that the cost is reduced, the electrodes can be used more easily by users, and the electrodes do not need to be frequently replaced or no conductive adhesive is needed.
(5) The system provided by the invention can fulfill multiple functions without various types of sensors and can fulfill a gesture recognition function and a blood pressure monitoring function only by means of a multiplexed electrode array, such that the sensor size, complexity and cost are reduced.
(6) The system provided by the invention carefully selects pulse impedance waveform features, including the slope, amplitude and time interval in different stages of one cardiac cycle, and a user-specific blood prediction module is obtained by neural network training.
(7) The system provided by the invention can quickly complete human-machine interaction by gestures, and a wearable smart wristband for monitoring the human blood pressure is provided.

### BRIEF DESCRIPTION OF THE DRAWINGS

FIG. 1 is a schematic diagram of a wristband device according to an embodiment.

FIG. 2 is a schematic diagram of the wristband device in an embodiment where the wristband device is worn correctly.

FIG. 3 is a schematic diagram of a wearable wristband formed by multiple wristband assemblies according to an embodiment.

FIG. 4 is a schematic structural design diagram of one wristband assembly according to an embodiment.

FIG. 5 is a logic block diagram of a multiplexing system for gesture recognition and pulse and blood pressure monitoring according to an embodiment.

FIG. 6 is a schematic diagram of excitation measurement modes in different functional modes according to an embodiment.

FIG. 7 is a schematic diagram of the software layout of a PC terminal according to an embodiment.

FIG. 8 is a schematic diagram of a neural network classifier used for gesture recognition of a PC terminal according to an embodiment.

FIG. 9 is a schematic diagram of a neural network regressor used for pulse-to-blood pressure conversion of a PC terminal according to an embedment.

Reference signs: 100, wearable bracket; 200, PCB; 300, display screen; 400, PC terminal; 110, hemispherical contact sensing electrode; 120, wristband assembly; 101, first contact sensing electrode; 102, second contact sensing electrode; 103, third contact sensing electrode; 104, fourth contact sensing electrode.

### DETAILED DESCRIPTION OF THE EMBODIMENTS

The technical solutions of the invention are described in detail below in conjunction with specific embodiments and accompanying drawings. Those skilled in the art can easily understand other advantages and effects of the invention with reference to the contents disclosed here. The invention can also be implemented or applied by means of other different specific embodiments. Based on different points of view and applications, various modifications or transformations can be made to details in the description without departing from the principle of the invention.

It should be noted that drawings in this embodiment illustrate the basic principle, component structures, operating process and effects of the invention merely by way of examples, only show components related to the invention, and are not plotted according to the number, form and size of components in actual implementation. In actual implementation, the form, number and scale of components can be changed, and the layout of the components may be more complex.

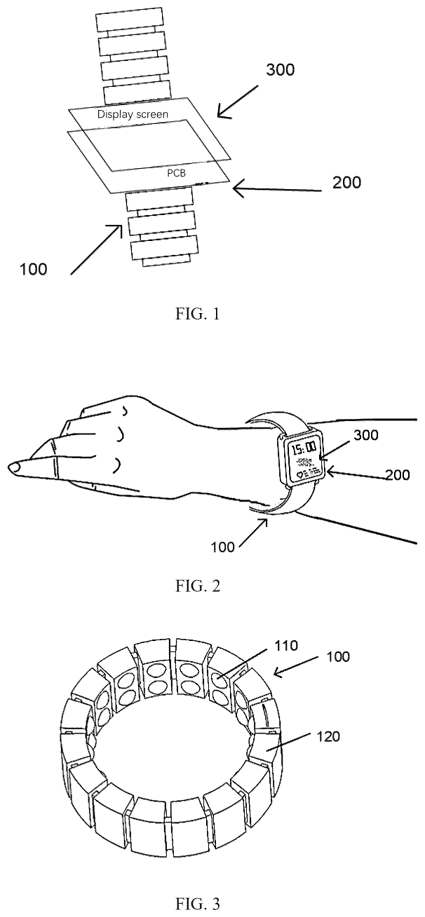

Referring to FIGS. 1-9, this embodiment provides a wristband device and system with a gesture recognition function and a continuous blood pressure monitoring function.

As shown in FIG. 1, a wristband device includes a wearable wristband 100, a PCB 200 and a display screen 300.

As shown in FIG. 2, the display screen 300 is fixed in a mechanical groove, the mechanical groove is seamlessly adhered to the top of the PCB 200 with a laminating adhesive to realize overlapped assembly, the display screen 300 is bonded to the PCB 200 and then arranged in a watchcase, and the wearable wristband 100 penetrates through the watchcase to be electrically connected to the PCB 200 by means of a wire. When used by users, the wristband device is disposed around the wrist of the users by means of the wearable wristband 100, and a visual surface of the display screen 300 faces the users.

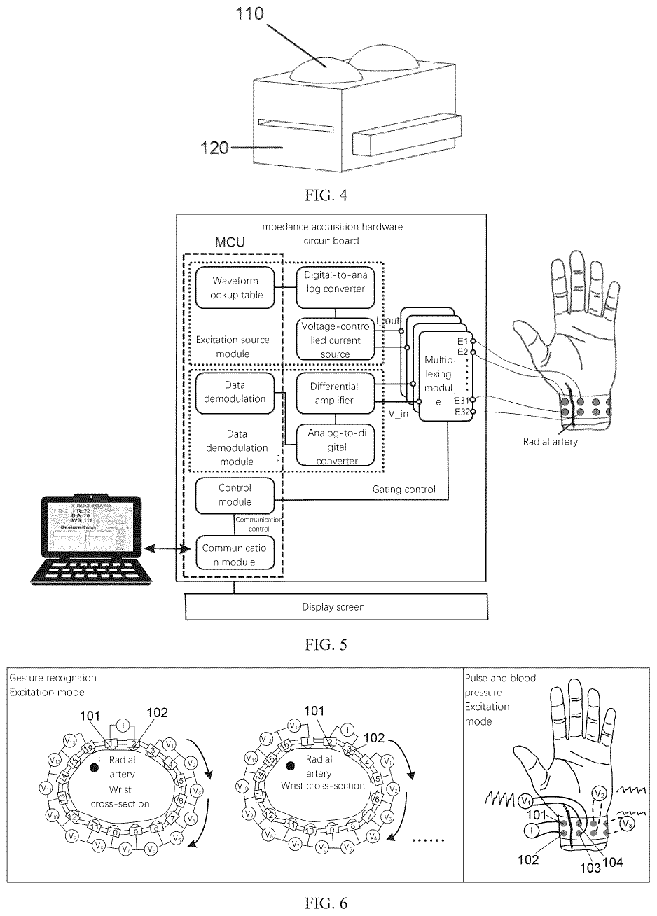

In this embodiment, as shown in FIG. 3, the wearable wristband 100 includes at least two wristband assemblies 120. As shown in FIG. 4, each wristband assembly 120 includes an assembly body and at least two contact sensing electrodes 110 arranged vertically, wherein the contact sensing electrodes are inlaid in an inner wall of the assembly body. The wristband assemblies 120 are connected by perforation or snap fit to form the wearable wristband 100 capable of encircling the wrist. Snap fit between adjacent wristband assemblies is realized by means of protrusion structures and groove structures on side faces of the assembly bodies, wherein the groove structures, not shown in FIG. 4, are located on side faces, opposite to the protrusion structures, of the assembly bodies. When the wearable wristband 100 is fastened on the wrist to measure electrical impedance, the position of the wearable wristband with respect to the wrist will not be changed, and during the measurement process, all the electrodes will be reliably in contact with user's skin to reduce interference.

The assembly body, in which the electrodes are inlaid, of the wearable wristband 100 is made from nylon, silicone or other suitable materials to fulfill a good fastening effect.

In this embodiment, a tag is arranged on the wearable wristband 100 and used for confirming electrodes located exactly above the radial artery to obtain an accurate impedance change signal of the radial artery.

The contact sensing electrodes, which function as bioimpedance sensing front-ends, have exposed surfaces facing the wrist and have a size of about 0.5 cm², may be configured to be in an excitation mode or an acquisition mode and used for injecting excitation currents to human wrists or measuring response voltages. In a gesture recognition functional mode, multiple electrodes of the wearable wristband are arranged in a ring array, and during measurement, the electrodes surround the wrist of a user in a circle. In a pulse and blood pressure monitoring functional mode, the wearable wristband includes at least two pairs of electrodes that are arranged vertically, and during measurement, the at least two pairs of electrodes arranged vertically are in close contact with skin above the radial artery that is about 2 cm below the styloid process of radius.

Specifically, in this embodiment, the wearable wristband 100 includes 16 wristband assemblies 120, each wristband assembly 120 includes two contact sensing electrodes 110 inlaid in the inner wall of the assembly body, the contact sensing electrodes of the wearable wristband 100 are arranged in two parallel circles with one above the other, and each circle includes 16 electrodes. Designers can select a suitable number of electrodes to function as sensing devices according to different requirements, and different electrode excitation and acquisition modes can be defined.

In this embodiment, the contact sensing electrodes 110 are hemispherical electrodes, such that the electrodes can be in good contact with the wrist, the wearing experience of users can be improved, and a wrist massage effect can be realized. According to different application scenarios, square electrodes, SMT electrodes or button electrodes may be adopted, and the invention has no limitation in this aspect.

In this embodiment, the contact sensing electrodes 110 are made from silver chloride-plated copper, which is low in cost, good in electrical conductivity, resistant to corrosion and harmless to humans. According to different requirements of designers, the contact sensing electrodes 110 may be made from other electrically conductive materials harmless to humans.

In this embodiment, the PCB 200, the specific structure of which is the same as an impedance acquisition hardware circuit board shown in FIG. 5, includes an excitation source module, a signal demodulation module, a multiplexing module, a control module, a communication module and a power supply module.

The excitation source module is used for injecting an excitation current into the electrodes in contact with the wrist, and the excitation current signal is a sinusoidal current signal with a selected frequency and within a safety current range. As shown in FIG. 5, the excitation source module obtains a unipolar sinusoidal voltage signal with an adjustable amplitude and frequency by means of a waveform lookup table, digital/analog signal conversion of the unipolar sinusoidal voltage signal is implemented by means of a digital-to-analog converter, an analog signal output by the digital-to-analog converter is input to a voltage-controlled current source, the voltage-controlled current source performs voltage/current transformation on the analog signal input thereto to obtain a differential current signal, and the differential current signal is injected to the contact sensing electrodes by means of the multiplexing module.

The signal demodulation module is used for extracting a response voltage signal fed back by the contact sensing electrodes in contact with the wrist. As shown in FIG. 5, the signal demodulation module acquires a response voltage signal of the contact sensing electrodes, the response voltage signal is a weak sinusoidal voltage signal at a same frequency as the excitation source module, differential/single-ended and analog/digital signal conversion of the response voltage signal is implemented by means of a differential amplifier and an analog-to-digital converter, noise is filtered out, and finally, an accurate bioelectrical impedance signal is extracted by means of a digital phase-sensitive demodulation module. In the gesture recognition functional mode, the signal demodulation module demodulates response voltage signals acquired by electrode pairs formed by the electrodes in all adjacent wristband assemblies into wrist impedance distribution data. In the pulse and blood pressure monitoring functional mode, the signal demodulation module demodulates a response voltage signal, that changes continuously with time, acquired by an electrode pair closest to the radial artery into pulse impedance waveform data of the radial artery in each cardiac cycle.

The multiplexing module is used for realizing gating of multiple contact sensing electrodes and switching between the excitation mode and the acquisition mode. More specifically, under the coordination of the control module, the multiplexing module switches a selected contact sensing electrode pair, receives a differential current output by the excitation source module in the excitation mode, and transmits an acquired response voltage signal to the signal demodulation signal in the acquisition mode, thus realizing multiplexing of multiple functional modes and multiple excitation modes. The multiplexing module may be formed by four 32-to-1 multiplexer chips, an analog switch chip or a relay, common terminals of the four multiplexer chips are respectively connected to two output terminals of the voltage-controlled current source and two input terminals of the differential amplifier, 32 gateable channels are connected to 32 electrodes, and an address line for controlling channel gating of the four multiplexer chips is connected to the control module.

The control module is used for coordinating tasks of other modules. More specifically, the control module is mainly used for controlling electrode gating of the multiplexing module, communication of the communication module with the display screen and a PC terminal, and start and stop of other modules.

The communication module is used for transmitting an acquired bioelectrical impedance signal to the PC terminal, receiving an operation result from the PC terminal, and transmitting the operation result from the PC to the display screen.

The power supply module, not shown in FIG. 5, is used for providing an operating voltage and power for full-load operation of the whole PCB.

More specifically, the modules of the impedance acquisition hardware circuit board may be specifically realized as follows:

the control module is typically configured in an MCU, a STM32 series MCU is used, or an MCU of other series or a FPGA is used as a main control chip to complete control and calculation of the device;

the communication module is realized by Bluetooth to guarantee the portability of the device, and the communication module may adopt other feasible communication schemes such as Ethernet communication.

A user control knob is arranged on a side face of the impedance acquisition hardware circuit board, and the user control knob is connected to the MCU and can control the multiplexing module by means of the MCU. A functional mode of the device can be selected by means of the user control knob, and the functional mode may be a gesture recognition functional mode or a pulse and blood pressure monitoring functional mode.

In this embodiment, the multiplexing module realizes gating of the contact sensing electrodes 110 and switching between excitation measurement modes. As shown in FIG. 6, the excitation measurement modes in different functional modes are as follows:
in the gesture recognition functional mode, an electrode pair is formed by electrodes selected from any two adjacent wristband assemblies of multiple wristband assemblies arranged around the wrist and is configured as an excitation current transmitter, electrode pairs formed by electrodes selected from the other adjacent wristband assemblies are configured as response voltage receivers for measuring a response voltage, and the electrode pair formed by electrodes selected from every two adjacent wristband assemblies is configured as the excitation current transmitter until an excitation current signal is applied to all the excitation current transmitters;
in the pulse and blood pressure monitoring functional mode, a pair of electrodes, a first contact sensing electrode 101 and a second contact sensing electrode 102, on one wristband assembly in the vicinity of the radial artery is selected to be configured as a differential excitation electrode pair, a differential excitation current is input to the differential excitation electrode pair, other electrode pairs, in the vicinity of the differential excitation electrode pair, on the same wristband assembly are configured to be in the acquisition mode to measure a response voltage, complex amplitudes V₁, V₂ and V₃ of a response voltage signal are demodulated to be used as bioimpedance representations of the radial artery and tissue around, the complex amplitudes are acquired continuously, the complex amplitudes of the voltage signal, that changes continuously with time, are taken as pulse and blood flow representation waveforms, amplitudes of pulse and blood flow representation waveforms obtained according to response voltage signals acquired by different response electrode pairs are compared, the response electrode pair corresponding a maximum amplitude and a maximum pulse wave signal peak-to-peak value is selected as an optimal acquisition electrode pair, for example, the pulse and blood flow representation waveform obtained according to the response voltage signal acquired by the response electrode pair formed by a third contact sensing electrode 103 and a fourth contact sensing electrode 104 has the maximum amplitude and the maximum pulse wave signal peak-to-peak value, and an alternating voltage, that changes continuously with time, of the response electrode pair formed by the third contact sensing electrode 103 and the fourth contact sensing electrode 104 is acquired continuously and used as an optimal response voltage signal.

In this embodiment, the display screen 300 is a small-sized liquid crystal display screen and used for displaying in real time the pulse waveform and heart rate of humans and a gesture recognition result or a monitored blood pressure calculated by the PC terminal. Specifically, the calculation result is obtained by Bluetooth communication with the communication module of the PCB.

This embodiment provides a system with a gesture recognition function and a continuous blood pressure monitoring function. As shown in FIG. 2, the system includes a wristband device and a cloud terminal, an APP terminal or a PC terminal 400 in wireless communication with the wristband device.

The cloud terminal, APP terminal or PC terminal 400 receives a bioelectrical impedance measurement signal detected by the wristband device and returns an operation result of a neural network arithmetic unit back to the wristband device.

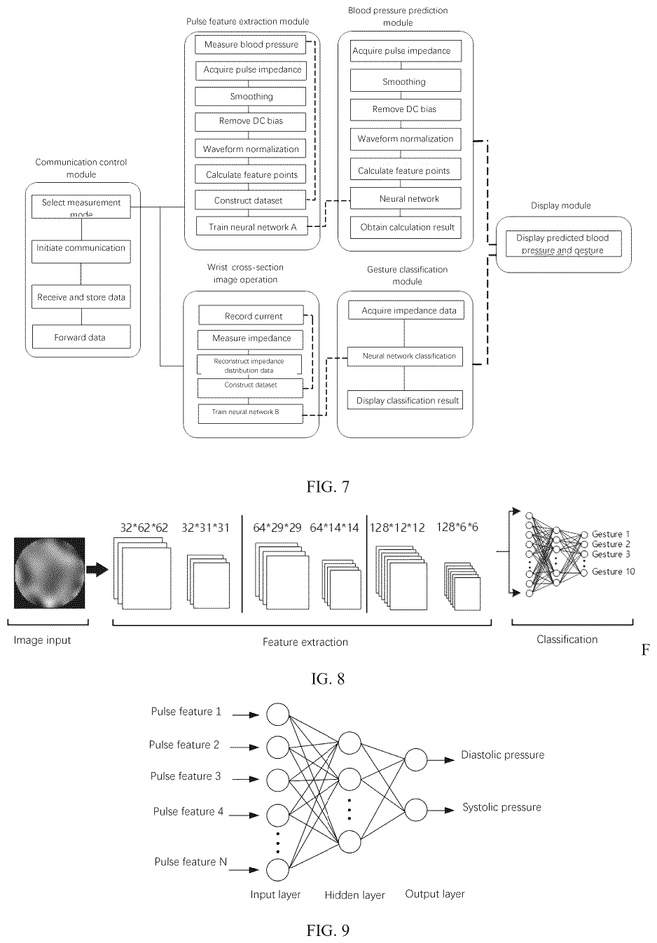

In this embodiment, the PC terminal 400 may be a common desktop computer, a laptop or a tablet personal computer, and the type of the PC terminal 400 is not limited. In a case where the system adopts the PC terminal 400, software modules shown in FIG. 7 are configured in the PC terminal 400.

Specifically, the software modules configured in the PC terminal 400 include a communication control module, a pulse feature extraction module, a blood pressure prediction module, a wrist cross-section image operation module, a gesture classification module and a result display module.

The communication control module is used for controlling communication between the PC terminal and the wristband device to be enabled or disabled, receiving bioelectrical impedance data transmitted from the wristband device, and transmitting gesture classification and blood pressure calculation results of the PC terminal 400 back to the wristband device to be displayed by the display screen 300.

The pulse feature extraction module is used for converting impedance waveform data of the radial artery into a pulse feature set, which is input to a neural network regressor to be used for blood pressure prediction.

The blood pressure prediction module is used for calibration and testing of blood pressure prediction, wherein when any one user uses the wristband device for the first time, blood pressure calibration needs to be performed by the following steps: acquiring and synchronously calibrating blood pressure information, calibrating the neural network regressor, and learning data features of the pulse feature set, wherein features of the pulse impedance waveform data of the radial artery in each cardiac cycle includes maximum slopes, impedance amplitudes and time intervals of a waveform segment from a starting point to a dominant wave peak of a pulse and blood flow representation waveform, a waveform segment from the dominant wave peak to a dicrotic wave trough of the pulse and blood flow representation waveform, a waveform segment from the dicrotic wave trough to an end point of the pulse and blood flow representation waveform, and an area defined by an impedance amplitude axis and a time axis, and all these features and a user blood pressure calibrated by an instrument at a same time when a radial artery pulse impedance signal is acquired are input together to a machine learning model for training; and after calibration is completed, converting, by the neural network regressor, pulse impedance data transmitted from the wristband device into a blood pressure, and displaying the blood pressure by a result display module of the PC terminal and the display screen 300 of the wristband device.

The wrist cross-section image operation module is used for converting wrist impedance distribution data from the wristband device into a wrist cross-sectional impedance change distribution image by means of an algorithm, wherein the wrist cross-sectional impedance change distribution image is input to a neural network classifier to be used for gesture recognition.

The gesture classification module is used for calibration and testing of gesture recognition, wherein when any one user uses the wristband device for the first time, gesture calibration needs to be performed by the following steps: acquiring and calibrating gesture information, calibrating the neural network classifier, learning wrist cross-sectional potential distribution image data features, after calibration is completed, classifying the wrist impedance distribution data transmitted from the wristband device as a gesture, and displaying a gesture classification result by the result display module of the PC terminal and the display screen 300 of the wristband device.

The result display module is used for visually displaying the bioelectrical impedance data from the wristband device and the gesture classification result and the blood pressure prediction result calculated by neural networks.

In this embodiment, the neural network arithmetic unit includes the neural network classifier used for gesture recognition and the neural network regressor used for blood pressure calculation. The neural network arithmetic unit is configured online or offline. When configured offline, the neural network arithmetic unit is configured in the PC terminal, a smartphone terminal, or a tablet computer terminal. When configured online, the neural network arithmetic unit is configured in a cloud terminal.

The neural network classifier is formed by three layers. As shown in FIG. 8, the neural network classifier specifically includes a cross-sectional image input layer, a hidden layer used for feature extraction, and a classification result output layer. Specifically, a processed wrist cross-sectional impedance change distribution image is input the neural network classifier, the hidden layer extracts image features, and after being trained, the neural network classifier can output a gesture recognition result by means of the classification result output layer.

The neural network regressor is formed by there layers. As shown in FIG. 9, the neural network regressor specifically includes a pulse feature input layer, a hidden layer and a prediction result output layer. Specifically, a selected pulse feature set and a blood pressure calibrated at the same time are input to the neural network regressor, dimension reduction is performed by the hidden layer, and then, a blood pressure prediction result is output by means of the prediction result output layer.

Neural networks used in this embodiment have a simple structure and a short calculation time and consumes a small quantity of hardware resources, such that the neural networks can be migrated into other electronic equipment integrated with an ASIC chip, a FPGA chip or an AI chip.

The basic principle, component structures, operating process and effects of the invention are described with reference to the above embodiments, but the application of the invention is not limited to the above description. Any skilled in the art can make embellishments or variations to the above embodiments without departing from the principle of the invention. Therefore, all equivalent embellishments or variations made by those ordinarily skilled in the art without departing from the principle of the invention should still fall within the protection scope of the invention and be covered by the claims of the invention.

What is claimed is:

1. A wristband device with a gesture recognition function and a continuous blood pressure monitoring function, comprising:
a wearable wristband, comprising at least two wristband assemblies, wherein each said wristband assembly comprises an assembly body and at least two contact sensing electrodes inlaid in an inner wall of the assembly body, the at least two contact sensing electrodes inlaid in the same assembly body are arranged vertically, and each said contact sensing electrode is configured to be in an excitation mode or an acquisition mode;
a PCB, used for realizing switching between a gesture recognition functional mode and a continuous blood pressure monitoring functional mode, applying an excitation current to an excitation current transmitter formed by two electrodes respectively selected from two adjacent said wristband assemblies and acquiring response voltage signals fed back by response voltage receivers formed by electrodes respectively selected from other adjacent said wristband assemblies in the gesture recognition functional mode, modulating the response voltage signals acquired in the gesture recognition functional mode into wrist impedance distribution data, applying the excitation current to an electrode pair on one said wristband assembly in a vicinity of a radial artery and acquiring response voltage signals fed back by electrode pairs on other said wristband assemblies in the continuous blood pressure monitoring functional mode, modulating an optimal response voltage acquired in the continuous blood pressure monitoring functional mode into pulse impedance waveform data of the radial artery in each cardiac cycle, transmitting the wrist impedance distribution data or the pulse impedance waveform data of the radial artery in each cardiac cycle to an upper computer, and receiving a gesture recognition and classification result or a predicted blood pressure fed back by the upper computer, and
a display screen, used for visualizing a heat rate of a user, the pulse impedance waveform data of the radial artery in each cardiac cycle and the gesture recognition and classification result or the predicted blood pressure received by the PCB.

2. The wristband device with the gesture recognition function and the continuous blood pressure monitoring function according to claim 1, wherein an optimal response voltage signal acquired in the continuous blood pressure monitoring functional mode is obtained as follows: complex amplitudes of the response voltage signals fed back by the electrode pairs on the other said wristband assemblies are extracted to be used as bioimpedance representations of the radial artery and a tissue around, the complex amplitudes of the response voltage signals fed back by the electrode pairs on the other said wristband assemblies are acquired continuously, the complex amplitudes of the response voltage signals that change continuously with time are taken as pulse and blood flow representation waveforms, amplitudes of the pulse and blood flow representation waveforms obtained according to the response voltage signals acquired by the electrode pairs on the other said wristband assemblies are compared, and the response voltage signal corresponding to the pulse and blood flow representation waveform with a maximum amplitude and a maximum pulse wave signal peak-to-peak value is selected as the optimal response voltage signal.

3. The wristband device with the gesture recognition function and the continuous blood pressure monitoring function according to claim 1, wherein the at least two wristband assemblies are connected by a perforation or a snap fit to form the wearable wristband capable of encircling a wrist, and the assembly body is made from, but not limited to, nylon or silicone; and the contact sensing electrodes are hemispherical electrodes, square electrodes, SMT electrodes or button electrodes.

4. The wristband device with the gesture recognition function and the continuous blood pressure monitoring function according to claim 1, wherein the PCB comprises:
an excitation source module, used for applying the excitation current to the contact sensing electrodes in the excitation mode;
a signal demodulation module, used for receiving response voltages fed back by the contact sensing electrodes in the acquisition mode, demodulating the response voltage signals acquired in the gesture recognition functional mode into the wrist impedance distribution data, and demodulating an optimal response voltage signal acquired in the continuous blood pressure monitoring functional mode into the pulse impedance waveform data of the radial artery in each cardiac cycle;
a multiplexing module, used for connecting any one said excitation current transmitter to the excitation source module and connecting the response voltage receivers to the signal demodulation module in the gesture recognition functional mode until the excitation current is applied to all the excitation current transmitters, and connecting the electrode pair of the wristband assembly in the vicinity of the radial artery to the excitation source module and connecting the electrode pairs of other wristband assemblies to the signal demodulation module in the continuous blood pressure monitoring functional mode;
a control module, used for controlling gating of channels in the multiplexing module, communication between a communication module and the display screen, and start and stop of the excitation source module and the signal demodulation module;
the communication module, used for transmitting the wrist impedance distribution data or the pulse impedance waveform data of the radial artery in each cardiac cycle to [[an]]the upper computer, receiving the gesture recognition and classification result or the predicted blood pressure fed back by the upper computer, and transmitting the gesture recognition and classification result or the predicted blood pressure to the display screen; and
a power supply module, used for providing an operating voltage and power for a full-load operation of the PCB.

5. The wristband device with the gesture recognition function and the continuous blood pressure monitoring function according to claim 4, wherein the excitation source module comprises:
a waveform lookup table, used for generating a unipolar sinusoidal voltage signal;
a digital-to-analog converter, used for converting the unipolar sinusoidal voltage signal into an analog signal and outputting the analog signal; and
a voltage-controlled current source, used for converting the analog signal output by the digital-to-analog converter into a differential current signal and outputting the differential current signal.

6. The wristband device with the gesture recognition function and the continuous blood pressure monitoring function according to claim 5, wherein the signal demodulation module comprises:
a differential amplifier, used for performing a differential amplification on the received response voltage signal and outputting a differential signal;
an analog-to-digital converter, used for converting the differential signal output by the differential amplifier into a single-ended signal, converting the single-ended signal into a digital signal and outputting the digital signal; and
a data demodulator, used for extracting the wrist impedance distribution data or the pulse impedance waveform data of the radial artery in each cardiac cycle from the digital signal output by the analog-to-digital converter.

7. The wristband device with the gesture recognition function and the continuous blood pressure monitoring function according to claim 6, wherein the multiplexing module is formed by four multiplexer chips, common terminals of the four multiplexer chips are respectively connected to two output terminals of the voltage-controlled current source and two input terminals of the differential amplifier, each said contact sensing electrode is electrically connected to a gateable channel of one said multiplexer chip, and an address line for controlling gating of the channels of the four multiplexer chips is connected to the control module.

8. The wristband device with the gesture recognition function and the continuous blood pressure monitoring function according to claim 1, wherein the display screen is fixed in a mechanical groove, the mechanical groove is seamlessly adhered to a top of the PCB with a laminating adhesive to realize an overlapped assembly, and the wearable wristband is electrically connected to the PCB.

9. A system with a gesture recognition function and a continuous blood pressure monitoring function, comprising the wristband device according to claim 1, and a PC terminal in a wireless communication with the wristband device, wherein the PC terminal comprises:
a communication control module, used for controlling the communication between the PC terminal and the wristband device to be enabled or disenabled, receiving the wrist impedance distribution data or the pulse impedance waveform data of the radial artery in each cardiac cycle transmitted from the wristband device, and transmitting the gesture recognition and classification result or the predicted blood pressure calculated by the PC terminal back to the wristband device;
a pulse feature extraction module, used for converting the pulse impedance waveform data of the radial artery in each cardiac cycle into pulse feature data to construct a pulse feature set;
a blood pressure prediction module, used for training a pre-deployed neural network regressor according to the pulse feature set, wherein the neural network regressor, after being trained, calculates the predicted blood pressure according to the pulse impedance waveform data of the radial artery in each cardiac cycle transmitted from the wristband device;
a wrist cross-section image operation module, used for converting the wrist impedance distribution data transmitted from the wristband device into a wrist cross-section impedance change distribution image;
a gesture classification module, used for training a pre-deployed neural network classifier according to the wrist cross-section impedance change distribution image, wherein the neural network classifier, after being trained, predicts the gesture recognition and classification result according to a real-time wrist impedance distribution data transmitted from the wristband device, and
a result display module, used for visualizing the wrist impedance distribution data or the pulse impedance waveform data of the radial artery transmitted from the wristband device, and visualizing the gesture classification result predicted by the gesture classification module or the predicted blood pressure calculated by the blood pressure prediction module.

10. The system with the gesture recognition function and the continuous blood pressure monitoring function according to claim 9, wherein the pulse feature set constructed by the pulse feature extraction module comprises the pulse feature data and a user blood pressure calibrated at a same time when the pulse impedance waveform data of the radial artery acquired, and the pulse feature data comprise, but not limited to, maximum slopes, impedance amplitudes and time intervals of a waveform segment from a starting point to a dominant wave peak of a pulse and blood flow representation waveform, a waveform segment from the dominant wave peak to a dicrotic wave trough of the pulse and blood flow representation waveform, a waveform segment from the dicrotic wave trough to an end point of the pulse and blood flow representation waveform, and an area defined by an impedance amplitude axis and a time axis.

11. The system with the gesture recognition function and the continuous blood pressure monitoring function according to claim 9, wherein in the wristband device, an optimal response voltage signal acquired in the continuous blood pressure monitoring functional mode is obtained as follows: complex amplitudes of the response voltage signals fed back by the electrode pairs on the other said wristband assemblies are extracted to be used as bioimpedance representations of the radial artery and a tissue around, the complex amplitudes of the response voltage signals fed back by the electrode pairs on the other said wristband assemblies are acquired continuously, the complex amplitudes of the response voltage signals that change continuously with time are taken as pulse and blood flow representation waveforms, amplitudes of the pulse and blood flow representation waveforms obtained according to the response voltage signals acquired by the electrode pairs on the other said wristband assemblies are compared, and the response voltage signal corresponding to the pulse and blood flow representation waveform with a maximum amplitude and a maximum pulse wave signal peak-to-peak value is selected as the optimal response voltage signal.

12. The system with the gesture recognition function and the continuous blood pressure monitoring function according to claim 9, wherein in the wristband device, the at least two wristband assemblies are connected by a perforation or a snap fit to form the wearable wristband capable of encircling a wrist, and the assembly body is made from, but not limited to, nylon or silicone; and the contact sensing electrodes are hemispherical electrodes, square electrodes, SMT electrodes or button electrodes.

13. The system with the gesture recognition function and the continuous blood pressure monitoring function according to claim 9, wherein in the wristband device, the PCB comprises:
an excitation source module, used for applying the excitation current to the contact sensing electrodes in the excitation mode;
a signal demodulation module, used for receiving response voltages fed back by the contact sensing electrodes in the acquisition mode, demodulating the response voltage signals acquired in the gesture recognition functional mode into the wrist impedance distribution data, and demodulating an optimal response voltage signal acquired in the continuous blood pressure monitoring functional mode into the pulse impedance waveform data of the radial artery in each cardiac cycle;
a multiplexing module, used for connecting any one said excitation current transmitter to the excitation source module and connecting the response voltage receivers to the signal demodulation module in the gesture recognition functional mode until the excitation current is applied to all the excitation current transmitters, and connecting the electrode pair of the wristband assembly in the vicinity of the radial artery to the excitation source module and connecting the electrode pairs of other wristband assemblies to the signal demodulation module in the continuous blood pressure monitoring functional mode;
a control module, used for controlling gating of channels in the multiplexing module, communication between a communication module and the display screen, and start and stop of the excitation source module and the signal demodulation module;
the communication module, used for transmitting the wrist impedance distribution data or the pulse impedance waveform data of the radial artery in each cardiac cycle to the upper computer, receiving the gesture recognition and classification result or the predicted blood pressure fed back by the upper computer, and transmitting the gesture recognition and classification result or the predicted blood pressure to the display screen; and
a power supply module, used for providing an operating voltage and power for a full-load operation of the PCB.

14. The system with the gesture recognition function and the continuous blood pressure monitoring function according to claim 13, wherein in the wristband device, the excitation source module comprises:
a waveform lookup table, used for generating a unipolar sinusoidal voltage signal;
a digital-to-analog converter, used for converting the unipolar sinusoidal voltage signal into an analog signal and outputting the analog signal; and
a voltage-controlled current source, used for converting the analog signal output by the digital-to-analog converter into a differential current signal and outputting the differential current signal.

15. The system with the gesture recognition function and the continuous blood pressure monitoring function according to claim 14, wherein in the wristband device, the signal demodulation module comprises:
a differential amplifier, used for performing a differential amplification on the received response voltage signal and outputting a differential signal;
an analog-to-digital converter, used for converting the differential signal output by the differential amplifier into a single-ended signal, converting the single-ended signal into a digital signal and outputting the digital signal; and
a data demodulator, used for extracting the wrist impedance distribution data or the pulse impedance waveform data of the radial artery in each cardiac cycle from the digital signal output by the analog-to-digital converter.

16. The system with the gesture recognition function and the continuous blood pressure monitoring function according to claim 15, wherein in the wristband device, the multiplexing module is formed by four multiplexer chips, common terminals of the four multiplexer chips are respectively connected to two output terminals of the voltage-controlled current source and two input terminals of the differential amplifier, each said contact sensing electrode is electrically connected to a gateable channel of one said multiplexer chip, and an address line for controlling gating of the channels of the four multiplexer chips is connected to the control module.

17. The system with the gesture recognition function and the continuous blood pressure monitoring function according to claim 9, wherein in the wristband device, the display screen is fixed in a mechanical groove, the mechanical groove is seamlessly adhered to a top of the PCB with a laminating adhesive to realize an overlapped assembly, and the wearable wristband is electrically connected to the PCB.
